(19) United States
(12) Patent Application Publication
(10) Pub. No.: US 2017/0348183 A1
(43) Pub. Date: Dec. 7, 2017

(54) MALE SEXUAL STIMULATION SYSTEMS

(71) Applicants: Kimrey Davis Tefft, Bluffton, SC (US); Larry Flynn, Hilton Head Island, SC (US)

(72) Inventors: Kimrey Davis Tefft, Bluffton, SC (US); Larry Flynn, Hilton Head Island, SC (US)

(21) Appl. No.: 15/608,953

(22) Filed: May 30, 2017

Related U.S. Application Data
(60) Provisional application No. 62/345,754, filed on Jun. 4, 2016.

(51) Int. Cl. A61H 19/00; A61F 5/41

(52) U.S. Cl. CPC A61H 19/32; A61H 19/00; A61F 5/41; A61H 2201/1692

(57) ABSTRACT

This disclosure includes a system for providing sexual stimulation to a male user. The system can include a balloon having a first inner side and a second inner side located opposite the first inner side. The system can also include a tube comprising a first end and a second end located opposite the first end. The first end can be coupled to the first inner side of the balloon and the second end can be coupled to the second inner side of the balloon. When the balloon is inflated, the first end of the tube can be arranged and configured to receive an erect penis of the male user.

### CROSS-REFERENCE TO RELATED APPLICATIONS

This application claims the benefit of U.S. Provisional Patent Application No. 62/345,754, filed Jun. 4, 2016; and entitled MALE SEXUAL STIMULATION SYSTEMS. The entire contents of U.S. Patent Application No. 62/345,754 are incorporated by reference herein.

### BACKGROUND

### Field

The present invention relates generally to sexual stimulation systems, and more particularly to sexual stimulation systems for male users.

### Description of Related Art

Oftentimes when a man is sexually aroused, he has sexual intercourse with a consenting partner. If a partner is unavailable, the man may masturbate to satisfy his arousal. Unfortunately, masturbation can require the use of lotion and tissues, and can often lead to a messy situation that can stain clothing. Moreover, masturbation does not closely simulate the feeling of sexual intercourse with a partner. Thus, there is a need for a system that reduces the chance of a mess and more closely simulates the feeling of sexual intercourse with a partner.

### SUMMARY

The present disclosure includes a system for providing sexual stimulation to a male user. The system can include a balloon having a first inner side and a second inner side located opposite the first inner side; and a tube comprising a first end and a second end located opposite the first end, the first end being coupled to the first inner side of the balloon and the second end being coupled to the second inner side of the balloon. When the balloon is inflated, the first end of the tube can be arranged and configured to receive an erect penis of the male user.

In some embodiments, the balloon comprises a mouth arranged and configured to receive a gas to thereby inflate the balloon. The balloon and the tube can be sized and shaped to receive the erect penis. The balloon can define an inflated state and a deflated state. When the balloon is in the inflated state, the tube can define a first length, and when the balloon is in the deflated state, the tube can define a second length that is less than the first length.

Additionally, in some embodiments, the tube comprises an inner surface comprising a plurality of ridges arranged and configured to stimulate the erect penis. The inner surface can comprise a lubricant arranged and configured to stimulate the erect penis.

Even still, in some embodiments, the tube and an inner portion of the balloon are arranged and configured to receive and retain ejaculate from the erect penis. When the balloon is deflated, the tube and the inner portion of the balloon can be arranged and configured to retain ejaculate from the erect penis.

In some embodiments, the balloon and tube are constructed from at least one of hypoallergenic latex and rubber. Also, some embodiments may include a package arranged and configured to receive the balloon when the balloon is in a deflated state.

The disclosure also includes a kit for providing sexual stimulation to a male user. The kit can include a first balloon having a first tube located within an inner portion of the first balloon, wherein when the first balloon is inflated, the first tube is arranged and configured to receive an erect penis of the male user. The kit can include a second balloon having a second tube located within an inner portion of the second balloon, wherein when the second balloon is inflated, the second tube is arranged and configured to receive the erect penis of the male user. The kit can also include a first package arranged and configured to receive the first balloon when the first balloon is in a deflated state; and a second package arranged and configured to receive the second balloon when the second balloon is in a deflated state.

In embodiments, the kit includes a third package arranged and configured to receive the first package and the second package. In some embodiments, the first balloon comprises a first mouth arranged and configured to receive gas to thereby inflate the first balloon, and the second balloon comprises a second mouth arranged and configured to receive gas to thereby inflate the second balloon.

The first tube can comprise an first inner surface comprising a first plurality of ridges arranged and configured to stimulate the erect penis. The second tube can comprise a second inner surface comprising a second plurality of ridges arranged and configured to stimulate the erect penis.

In some embodiments, the kit further includes a lubricant arranged and configured to stimulate the erect penis. The first tube and the inner portion of the first balloon can be arranged and configured to receive and retain ejaculate from the erect penis. Additionally, the second tube and the inner portion of the second balloon can be arranged and configured to receive and retain ejaculate from the erect penis.

Even still, in some embodiments, when the first balloon is deflated, the first tube and the inner portion of the first balloon are arranged and configured to retain ejaculate from the erect penis, and when the second balloon is deflated, the second tube and the inner portion of the second balloon are arranged and configured to retain ejaculate from the erect penis. In some embodiments, the first balloon, first tube, second balloon, and second tube are constructed from at least one of hypoallergenic latex and rubber.

In some embodiments, the first package defines a surface area less than or equal to 36 centimeters squared, and the second package defines a surface area less than or equal to 36 centimeters. The third package can define a surface area greater than or equal to 36 centimeters squared.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a perspective view of a stimulation system, according to some embodiments.

FIG. 2 illustrates a perspective view of another stimulation system, according to some embodiments.

FIGS. 3a and 3b illustrate a perspective view of a stimulation system in an inflated state and a deflated state, according to some embodiments.

FIG. 4 illustrates a perspective view of a stimulation system having ridges, according to some embodiments.

FIG. 5 illustrates a perspective view of a stimulation system in a deflated state and a package to receive the stimulation system, according to some embodiments.

FIG. 6 illustrates a kit for providing sexual stimulation, according to some embodiments.

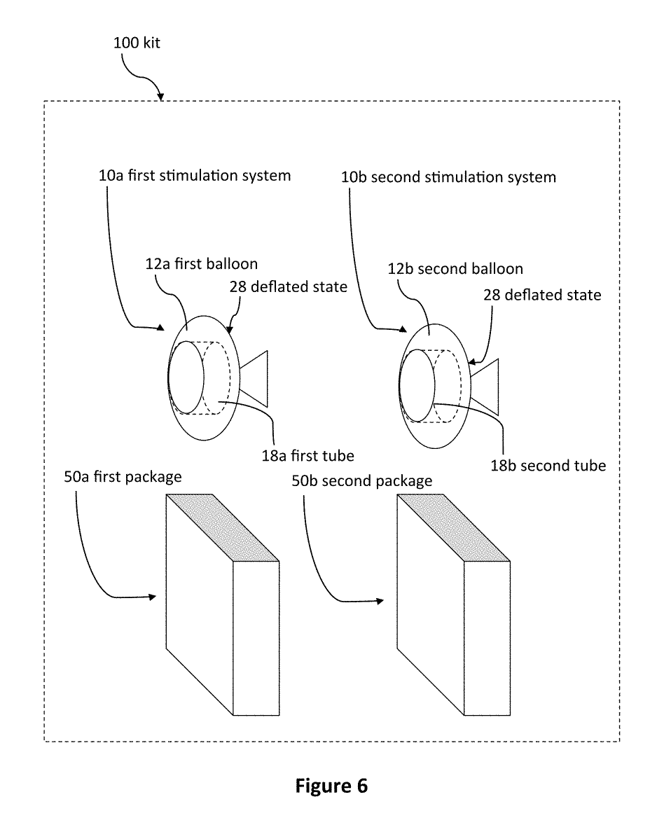

FIG. 7 illustrates a perspective view of a first package, a second package, and a third package, according to some embodiments.

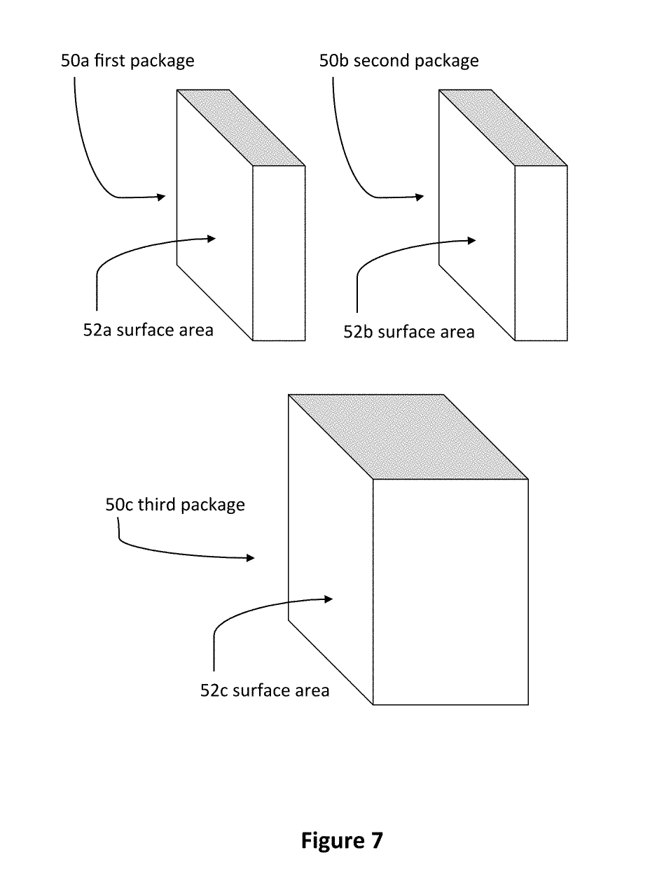

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### LIST OF REFERENCE NUMERALS

10—Stimulation system
12—Balloon
14—First inner side
16—Second inner side
18—Tube
20—First end
22—Second end
24—Mouth
26—Inflated state
28—Deflated state
30—First length
32—Second length
40—Inner surface
42—Plurality of ridges
44—Lubricant
46—Ejaculate
50—Package
52—Surface area
100—Kit

### Introduction

When men are sexually aroused they may uses devices or gadgets to assist with the ejaculation process. Unfortunately, many men dislike going to sex shops or making purchases online to buy gadgets for sexual stimulation. Accordingly, there is a need for a system to be readily available and at an affordable price. The system should be a clean and provide an easy-to-use option. The system can provide a safe and private way for the man to pleasure himself without the need for or risk of sexual contact with another person.

Accordingly, the present disclosure describes a disposable, sexual stimulation system for men who want a more socially acceptable and readily available solution at an affordable price. The stimulation system could be sold wherever condoms are sold, such as drug stores, grocery stores, gas stations, truck stops, and the like. The system may appeal to traveling businessmen, truck drivers, inmates, men with partners who are ill or otherwise unable to have sex, military men who have to be away from their partners for extended periods of time, and the like.

### Stimulation System Embodiments

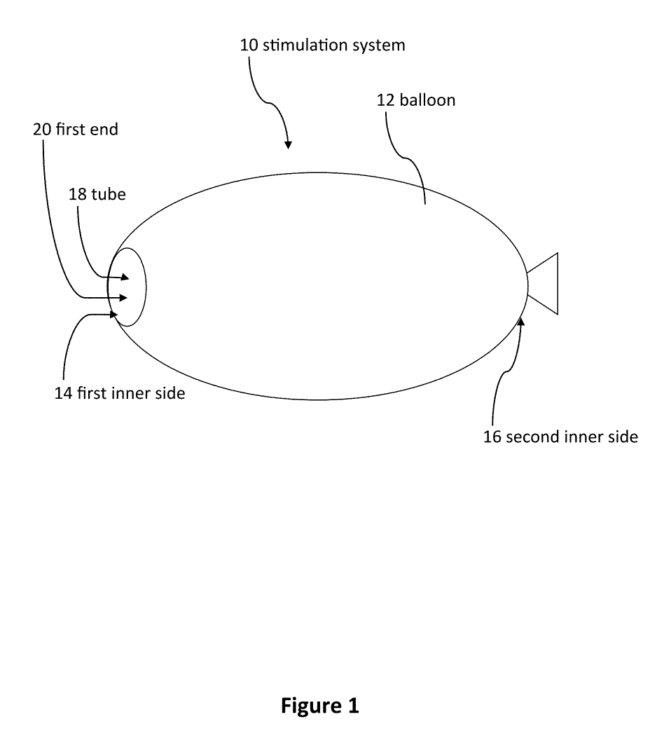

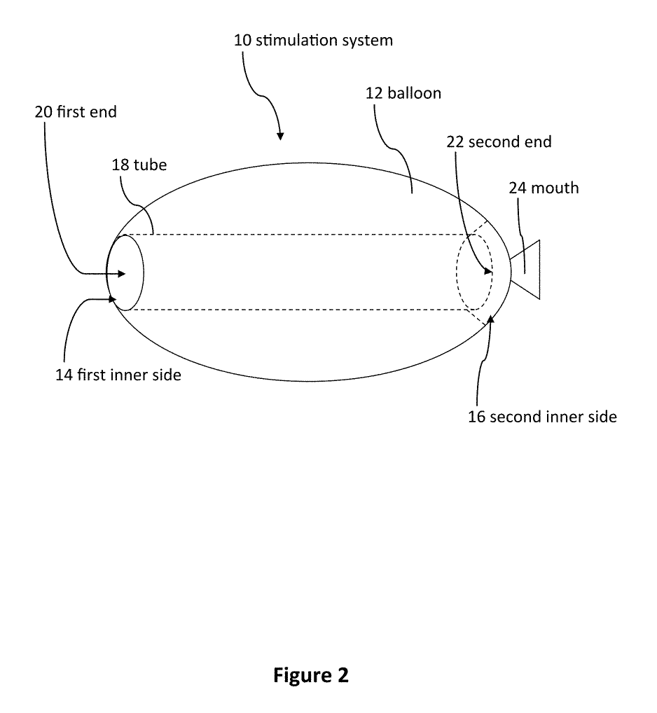

As shown in FIGS. 1 and 2, a system 10 for providing sexual stimulation to a male user can include a balloon 12 having a first inner side 14 and a second inner side 16 located opposite the first inner side 14. The system can also include a tube 18 comprising a first end 20 and a second end 22 located opposite the first end 20. The first end 20 can be coupled to the first inner side 14 of the balloon 12. Accordingly, the second end 22 can be coupled to the second inner side 16 of the balloon 12.

When the balloon 12 is inflated, the first end 20 of the tube 18 can be arranged and configured to receive an erect penis of the male user. The balloon 12 and the tube 18 can be sized and shaped to receive the erect penis of a male user. In some embodiments, the balloon 12 and 18 are sized and shaped to receive an erect penis ranging in length from 1 inch to 16 inches. In many embodiments, the balloon 12 and tube 18 are constructed from hypoallergenic latex, rubber, and the like.

As shown in FIG. 2, in some embodiments, the balloon comprises a mouth 24 that is arranged and configured to receive a gas to thereby inflate the balloon 12. It should be appreciated that the mouth 24 can be the mouth of a traditional balloon that receives gas, such as carbon dioxide and air from the lips of a person who inflates the balloon.

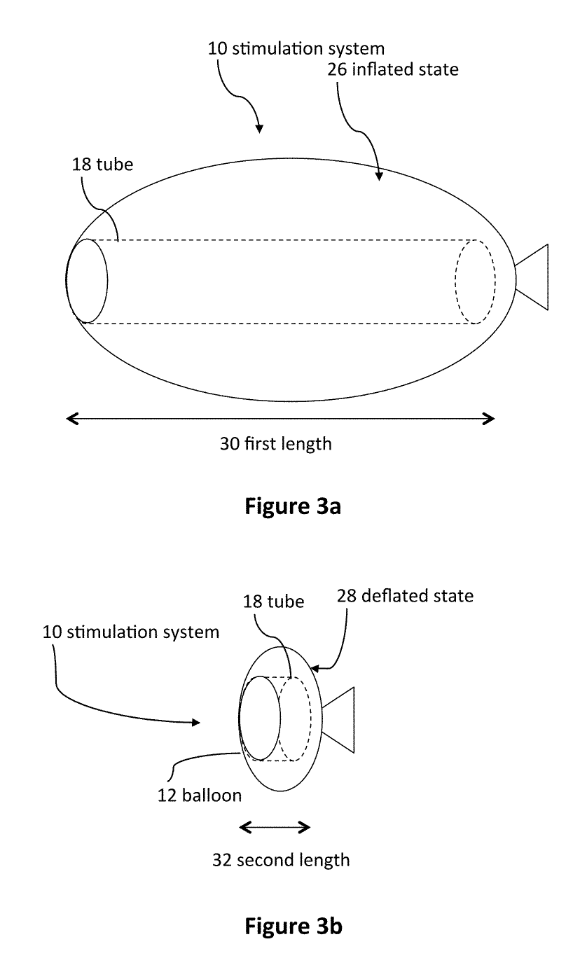

As illustrated in FIGS. 3a and 3b, the balloon 12 can define an inflated state 26 and a deflated state 28. When the balloon 12 is in the inflated state 26, the tube 18 can define a first length 30, and when the balloon 12 is in the deflated state 28, the tube 18 can define a second length 32 that is less than the first length 30.

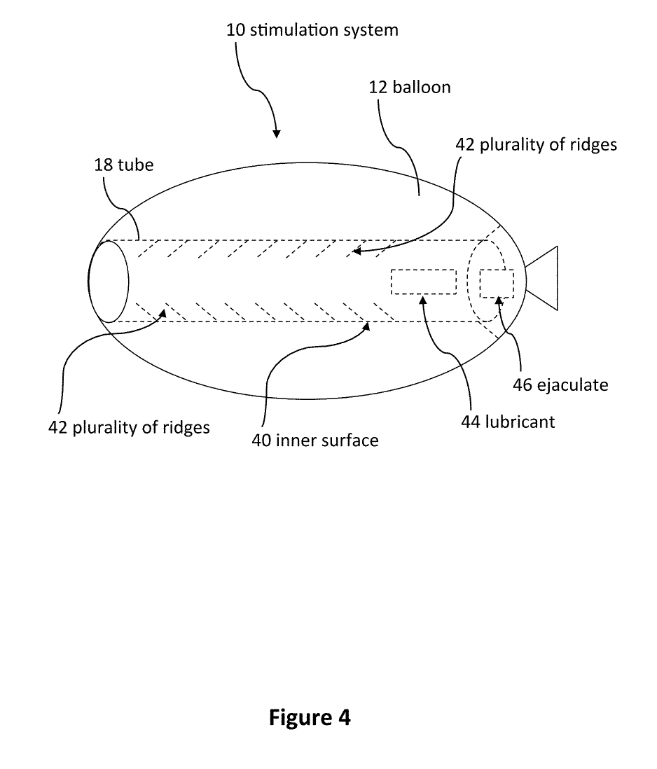

Now with reference to FIG. 4, the tube can comprise an inner surface 40 comprising a plurality of ridges 42 arranged and configured to stimulate the erect penis. In some embodiments, the plurality of ridges can be the same structure and appearance as ridges found on the external surface of a conventional condom. In this manner, the male user can move the system 10 up and down along his erect penis to thereby stimulate ejaculation.

Embodiments may include additional features to enhance the experience for the male user. For example, the inner surface 40 may comprise a lubricant 44 arranged and configured to further stimulate the erect penis. Accordingly, the tube 18 and an inner portion of the balloon 12 can be arranged and configured to receive and retain ejaculate 46 from the erect penis. The system 10 can retain the ejaculate 46 whether the balloon 12 is in the inflated state or the deflated state.

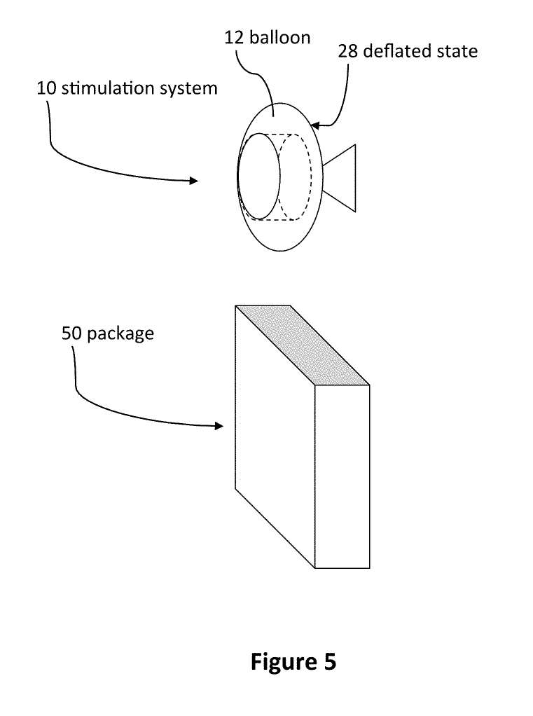

As shown in FIG. 5, the system 10 can also include a package 50 arranged and configured to receive the balloon 12 when the balloon 12 is in a deflated state 28. In some embodiments, the package 50 is the same packaging used for conventional condoms. For example, the package 50 can be the same form factor as that of a conventional condom wrapper. In this manner, the package 50 can be sold through the same sales channels as conventional condoms, such as condom vending machines, point-of-sale display stands, and the like.

The disclosure also includes a kit 100 for providing sexual stimulation to a male user. In some embodiments, the kit 100 includes a first balloon 12a having a first tube 18a located within an inner portion of the first balloon 12a. When the first balloon 12a is inflated, the first tube 18a can be arranged and configured to receive an erect penis of the male user. Additionally, the kit 100 can also include a second balloon 12b having a second tube 18b located within an inner portion of the second balloon 12b. Like the first balloon 12a, when the second balloon 12b is inflated, the second tube 18b can be arranged and configured to receive the erect penis of the male user.

The kit 100 can also include a first package 50a arranged and configured to receive the first balloon 12a when the first balloon 12a is in a deflated state 28, and a second package 50b arranged and configured to receive the second balloon 12b when the second balloon 12b is in a deflated state 28. In some embodiments, the kit 100 also includes a third package 50c arranged and configured to receive the first package 50a and the second package 50b. In this manner, the third package 50c can be sized and shaped to receive multiple first and second packages 50a, 50b.

In some embodiments, the first package 50a defines a surface area less than or equal to 36 centimeters squared, and the second package 50b defines a surface area less than or equal to 36 centimeters. Additionally, in some embodiments, the third package 50c defines a surface area greater than or equal to 36 centimeters squared. In this manner, the kit 100 can be sold through retail channels as a multi-pack system.

### Interpretation

None of the steps or limitations described herein is essential or indispensable. Any of the steps or limitations can be adjusted or modified. Other or additional steps and/or limitations can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computer, processor, or machine configured to execute computer instructions. The code modules may be stored on any type of non-transitory computer-readable storage medium or tangible computer storage device, such as a hard drive solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. A system for providing sexual stimulation to a male user, comprising:
a balloon having a first inner side and a second inner side located opposite the first inner side; and
a tube comprising a first end and a second end located opposite the first end, the first end being coupled to the first inner side of the balloon and the second end being coupled to the second inner side of the balloon,
wherein when the balloon is inflated, the first end of the tube is arranged and configured to receive an erect penis of the male user.

2. The system of claim 1, wherein the balloon comprises a mouth arranged and configured to receive a gas to thereby inflate the balloon.

3. The system of claim 2, wherein the balloon and the tube are sized and shaped to receive the erect penis.

4. The system of claim 3, wherein the balloon defines an inflated state and a deflated state, and wherein when the balloon is in the inflated state, the tube defines a first length, and when the balloon is in the deflated state, the tube defines a second length that is less than the first length.

5. The system of claim 2, wherein the tube comprises an inner surface comprising a plurality of ridges arranged and configured to stimulate the erect penis.

6. The system of claim 5, wherein the inner surface comprises a lubricant arranged and configured to stimulate the erect penis.

7. The system of claim 2, wherein the tube and an inner portion of the balloon are arranged and configured to receive and retain ejaculate from the erect penis.

8. The system of claim 7, wherein when the balloon is deflated, the tube and the inner portion of the balloon are arranged and configured to retain ejaculate from the erect penis.

9. The system of claim 2, wherein the balloon and tube are constructed from at least one of hypoallergenic latex and rubber.

10. The system of claim 2, further comprising a package arranged and configured to receive the balloon when the balloon is in a deflated state.

11. A kit for providing sexual stimulation to a male user, comprising:
a first balloon having a first tube located within an inner portion of the first balloon, wherein when the first balloon is inflated, the first tube is arranged and configured to receive an erect penis of the male user;
a second balloon having a second tube located within an inner portion of the second balloon, wherein when the second balloon is inflated, the second tube is arranged and configured to receive the erect penis of the male user;
a first package arranged and configured to receive the first balloon when the first balloon is in a deflated state; and
a second package arranged and configured to receive the second balloon when the second balloon is in a deflated state.

12. The kit of claim 11, further comprising a third package arranged and configured to receive the first package and the second package.

13. The kit of claim 12, wherein the first balloon comprises a first mouth arranged and configured to receive gas to thereby inflate the first balloon, and the second balloon comprises a second mouth arranged and configured to receive gas to thereby inflate the second balloon.

14. The kit of claim 12, wherein the first tube comprises an first inner surface comprising a first plurality of ridges arranged and configured to stimulate the erect penis, and the second tube comprises a second inner surface comprising a second plurality of ridges arranged and configured to stimulate the erect penis.

15. The kit of claim 12, further comprising a lubricant arranged and configured to stimulate the erect penis.

16. The kit of claim 12, wherein the first tube and the inner portion of the first balloon are arranged and configured to receive and retain ejaculate from the erect penis, and the second tube and the inner portion of the second balloon are arranged and configured to receive and retain ejaculate from the erect penis.

17. The kit of claim 16, wherein when the first balloon is deflated, the first tube and the inner portion of the first balloon are arranged and configured to retain ejaculate from the erect penis, and when the second balloon is deflated, the second tube and the inner portion of the second balloon are arranged and configured to retain ejaculate from the erect penis.

18. The kit of claim 12, wherein the first balloon, first tube, second balloon, and second tube are constructed from at least one of hypoallergenic latex and rubber.

19. The kit of claim 12, wherein the first package defines a surface area less than or equal to 36 centimeters squared, and the second package defines a surface area less than or equal to 36 centimeters.

20. The kit of claim 19, wherein the third package defines a surface area greater than or equal to 36 centimeters squared.
